(19) United States
(12) Patent Application Publication
(10) Pub. No.: US 2024/0115794 A1
(43) Pub. Date: Apr. 11, 2024

(54) SYSTEMS AND METHODS FOR PAIRING WITH WOUND THERAPY DEVICES

(71) Applicant: T.J.Smith and Nephew,Limited, Hull (GB)

(72) Inventor: Felix Clarence Quintanar, Hull (GB)

(21) Appl. No.: 17/768,416

(22) PCT Filed: Oct. 12, 2020
(86) PCT No.: PCT/EP2020/078551

§ 371 Date: Apr. 12, 2022

(30) Foreign Application Priority Data
Oct. 14, 2019 (GB) 1914846.9

(51) Int. Cl. A61M 1/00; G16H 40/63

(52) U.S. Cl. CPC A61M 1/91; G16H 40/63; A61M 2205/3576

(57) ABSTRACT

A negative pressure wound therapy device can include a housing with an exterior surface including a region configured to facilitate pairing with a computing device. Electronic circuitry of the negative pressure wound therapy device can be configured to receive pairing data from the computing device in response to the computing device physically contacting a portion of the exterior surface of the housing within the region or the computing device being positioned at a distance satisfying a threshold distance from the exterior surface of the housing within the region, the pairing data being received using a first communication protocol, pair with the computing device using the pairing data, and subsequent to the pairing, transmit data to and receive data from the computing device using a second communication protocol different from the first communication protocol.

### TECHNICAL FIELD

Embodiments described herein relate to apparatuses, systems, and methods the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy device can include a housing with an exterior surface. Exterior surface of the housing can include a region configured to facilitate pairing with a computing device. The housing can enclose a source of negative pressure and electronic circuitry. The source of negative pressure can be configured to aspirate fluid from a wound covered by a wound dressing. Electronic circuitry can be configured to control the source of negative pressure and to wirelessly transmit and receive data. Electronic circuitry can be configured to receive pairing data from the computing device in response to the computing device physically contacting a portion of the exterior surface of the housing within the region or the computing device being positioned at a distance satisfying a threshold distance from the exterior surface of the housing within the region. Pairing data can be received using a first communication protocol. Electronic circuitry can be configured to pair with the computing device using the pairing data. Electronic circuitry can be configured to, subsequent to the pairing, transmit data to and receive data from the computing device using a second communication protocol different from the first communication protocol.

The negative pressure wound therapy device of any of the preceding paragraphs and/or any of the negative pressure wound therapy devices described herein can include one or more of the following features. Electronic circuitry can be configured to receive the pairing data in response to the computing device physically contacting the portion of the exterior surface of the housing within the region or the computing device being positioned at the distance from the exterior surface of the housing within the region satisfying the threshold distance and not in response to the computing device physically contacting any other portion of the exterior surface of the housing or the computing device being positioned at another distance from the exterior surface of the housing within the region not satisfying the threshold distance.

The negative pressure wound therapy device of any of the preceding paragraphs and/or any of the negative pressure wound therapy devices described herein can include one or more of the following features. The device can include an antenna coupled to the electronic circuitry and configured to wirelessly transmit and receive data. Coverage area of the antenna can include one or more of the region of the exterior surface of the housing or a volume extending no farther than the threshold distance away from the exterior surface of the housing within the region. Coverage area of the antenna may not extend to any other portion of the exterior surface of the housing outside of the region or to any other location outside the volume. Antenna can be positioned within the housing adjacent to the exterior surface of the housing within the region. Exterior surface of the housing within the region can be thinner than exterior surface of the housing adjacent to the region. The device can include an electromagnetic shield that limits the coverage area of the antenna to one or more of the region of the exterior surface of the housing or the volume extending no farther than the threshold distance away from the exterior surface of the housing within the region.

The negative pressure wound therapy device of any of the preceding paragraphs and/or any of the negative pressure wound therapy devices described herein can include one or more of the following features. Electronic circuitry can be configured to transmit data to and receive data from the computing device only subsequent to the pairing with the computing device. Pairing data can include a passcode or an IP address. Electronic circuitry is can be configured to encrypt data transmitted over the second communication protocol using the paring data. First communication protocol can include near-field communication (NFC) or radio frequency identification (RFID). Second communication protocol can include Bluetooth or Wi-Fi. Data transmitted to the computing device can include one or more of operational data associated with provision of negative pressure wound therapy or location data associated with location of the device. Computing device can include another negative pressure wound therapy device.

The negative pressure wound therapy device of any of the preceding paragraphs and/or any of the negative pressure wound therapy devices described herein can include one or more of the following features. The device can include an optical sensor positioned within the housing adjacent to the exterior surface of the housing within the region. Exterior surface of the housing within the region can be least one of optically transparent or optically translucent. Exterior surface of the housing adjacent to the region can be optically opaque. First communication protocol can include an optical communication protocol. Exterior surface of the housing within the region can include a window made of optically transparent material. Exterior surface of the housing can include a light transmitter. First communication protocol can include an optical communication protocol. Exterior surface of the housing can include a barcode. Barcode can include the pairing data.

A negative pressure wound therapy system can include the negative pressure wound therapy device of any of the preceding paragraphs and/or any of the negative pressure wound therapy devices described herein and a wound dressing.

A method of operating a negative pressure wound therapy device can include receiving pairing data from a remote computing device in response to the remote computing device physically contacting a portion of an exterior surface of a housing of the negative pressure wound therapy device within a region configured to facilitate pairing with the remote computing device or the remote computing device being positioned at a distance satisfying a threshold distance from the exterior surface of the housing within the region. Pairing data can be received using a first communication protocol. The method can include pairing with the remote computing device using the pairing data. The method can include, subsequent to the pairing, transmitting data to and receiving data from the remote computing device using a second communication protocol different from the first communication protocol. The method of this paragraph and/or any of the methods described herein can be performed by electronic circuitry of the negative pressure wound therapy device.

The method of any of the preceding paragraphs and/or any of the methods described herein can include one or more of the following features. The method can include, by the electronic circuitry, receiving the pairing data in response to the remote computing device physically contacting the portion of the exterior surface of the housing within the region or the remote computing device being positioned at the distance from the exterior surface of the housing within the region satisfying the threshold distance and not in response to the remote computing device physically contacting any other portion of the exterior surface of the housing or the remote computing device being positioned at another distance from the exterior surface of the housing within the region not satisfying the threshold distance.

The method of any of the preceding paragraphs and/or any of the methods described herein can include one or more of the following features. Negative pressure wound therapy device can include an antenna with a coverage area including one or more of the region of the exterior surface of the housing or a volume extending no farther than the threshold distance away from the exterior surface of the housing within the region. Coverage area of the antenna may not extend to any other portion of the exterior surface of the housing outside of the region or to any other location outside the volume. Antenna can be positioned within the housing adjacent to the exterior surface of the housing within the region. Exterior surface of the housing within the region can be thinner than exterior surface of the housing adjacent to the region. Antenna can be at least partially shielded by an electromagnetic shield that limits the coverage area of the antenna to one or more of the region of the exterior surface of the housing or the volume extending no farther than the threshold distance away from the exterior surface of the housing within the region.

The method of any of the preceding paragraphs and/or any of the methods described herein can include one or more of the following features. Negative pressure wound therapy device can include an optical sensor positioned within the housing adjacent to the exterior surface of the housing within the region. Exterior surface of the housing within the region can be at least one of optically transparent or optically translucent. Exterior surface of the housing adjacent to the region can be optically opaque. First communication protocol can include an optical communication protocol. A barcode can be positioned on the exterior surface of the housing. The barcode can include the pairing data.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1 illustrates a reduced pressure wound therapy system.

FIGS. 2A-2B illustrate a reduced pressure wound therapy device and canister.

FIG. 3 illustrates a schematic of a reduced pressure wound therapy device.

FIGS. 4A-4C illustrate a reduced pressure wound therapy device.

FIG. 5 illustrates a process of pairing with a therapy device.

### DETAILED DESCRIPTION

Embodiments disclosed herein relate to systems and methods of monitoring and/or treating a wound. It will be appreciated that throughout this specification reference is made to a wound. It is to be understood that the term wound is to be broadly construed and encompasses open and closed wounds in which skin is torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, and/or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760—X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than —X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat and/or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level and/or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, and/or methods of efficiently adjusting TNP prescriptions and delivering effective TNP therapy.

### Negative Pressure System

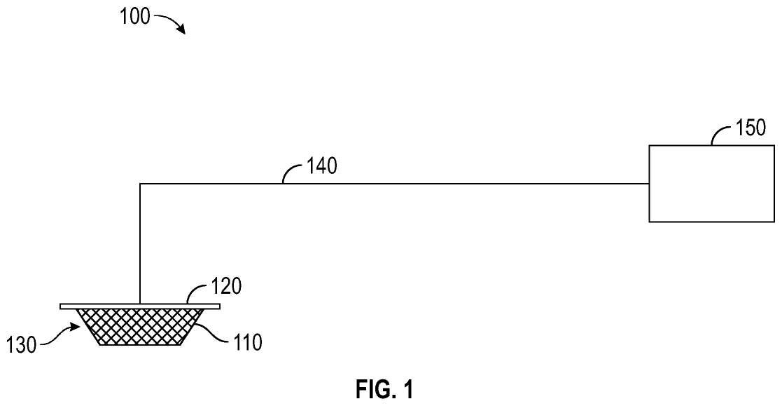

FIG. 1 illustrates a negative or reduced pressure wound treatment (or TNP) system 100 comprising a wound filler 130 placed inside a wound cavity 110, the wound cavity sealed by a wound cover 120. The wound filler 130 in combination with the wound cover 120 can be referred to as wound dressing. A single or multi lumen tube or conduit 140 is connected the wound cover 120 with a reduced (or negative) pressure wound therapy device 150 configured to supply reduced pressure. The wound cover 120 can be in fluidic communication with the wound cavity 110. With any of the systems disclosed herein, as is illustrated in FIG. 1, a negative pressure wound therapy device (sometimes as a whole or partially referred to as a “pump assembly”) can be a canisterless (meaning that exudate is collected in the wound dressing or is transferred via tube 140 for collection to another location). However, any of the pump assemblies disclosed herein can be configured to include or support a canister. Additionally, with any of the systems disclosed herein, any of the pump assemblies can be mounted to or supported by the dressing, or adjacent to the dressing. The wound filler 130 can be any suitable type, such as hydrophilic or hydrophobic foam, gauze, inflatable bag, and so on. The wound filler 130 can be conformable to the wound cavity 110 such that it substantially fills the cavity. The wound cover 120 can provide a substantially fluid impermeable seal over the wound cavity 110. In some cases, the wound cover 120 has a top side and a bottom side, and the bottom side adhesively (or in any other suitable manner) seals with wound cavity 110. The conduit 140 or any other conduit disclosed herein can be formed from polyurethane, PVC, nylon, polyethylene, silicone, or any other suitable material.

The wound cover 120 can have a port (not shown) configured to receive an end of the conduit 140. In some cases, the conduit 140 can otherwise pass through and/or under the wound cover 120 to supply reduced pressure to the wound cavity 110 so as to maintain a desired level of reduced pressure in the wound cavity. The conduit 140 can be any suitable article configured to provide at least a substantially sealed fluid flow pathway or path between the pump assembly 150 and the wound cover 120, so as to supply the reduced pressure provided by the pump assembly 150 to wound cavity 110.

The wound cover 120 and the wound filler 130 can be provided as a single article or an integrated single unit. In some cases, no wound filler is provided and the wound cover by itself may be considered the wound dressing. The wound dressing may then be connected, via the conduit 140, to a source of negative pressure, such as the pump assembly 150. In some cases, though not required, the pump assembly 150 can be miniaturized and portable, although larger conventional pumps such can also be used.

The wound cover 120 can be located over a wound site to be treated. The wound cover 120 can form a substantially sealed cavity or enclosure over the wound site. In some cases, the wound cover 120 can be configured to have a film having a high water vapour permeability to enable the evaporation of surplus fluid, and can have a superabsorbing material contained therein to safely absorb wound exudate. It will be appreciated that throughout this specification reference is made to a wound. In this sense it is to be understood that the term wound is to be broadly construed and encompasses open and closed wounds in which skin is torn, cut or punctured or where trauma causes a contusion, or any other surficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, acute wounds, chronic wounds, surgical incisions and other incisions, subacute and dehisced wounds, traumatic wounds, flaps and skin grafts, lacerations, abrasions, contusions, burns, diabetic ulcers, pressure ulcers, stoma, surgical wounds, trauma and venous ulcers or the like. In some cases, the components of the TNP system described herein can be particularly suited for incisional wounds that exude a small amount of wound exudate.

The system can be designed to operate without the use of an exudate canister. The system can be configured to support an exudate canister. In some cases, configuring the pump assembly 150 and tubing 140 so that the tubing 140 can be quickly and easily removed from the pump assembly 150 can facilitate or improve the process of dressing or pump changes, if necessary. Any of the pump assemblies disclosed herein can be configured to have any suitable connection between the tubing and the pump.

The pump assembly 150 can be configured to deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively a pressure range of up to −75 mmHg, up to −80 mmHg or over −80 mmHg can be used. Also in some cases a pressure range of below −75 mmHg can be used. Alternatively a pressure range of over approximately −100 mmHg, or even 150 mmHg, can be supplied by the pump assembly 150.

The pump assembly 150 can be configured to provide continuous or intermittent negative pressure therapy. Continuous therapy can be delivered at above −25 mmHg, −25 mmHg, −40 mmHg, −50 mmHg, −60 mmHg, −70 mmHg, −80 mmHg, −90 mmHg, −100 mmHg, −120 mmHg, −140 mmHg, −160 mmHg, −180 mmHg, −200 mmHg, or below −200 mmHg. Intermittent therapy can be delivered between low and high negative pressure set points. Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values. The first and second durations can be selected from the following range: less than 2 minutes, 2 minutes, 3 minutes, 4 minutes, 6 minutes, 8 minutes, 10 minutes, or greater than 10 minutes. In some cases, switching between low and high set points and vice versa can be performed according to a step waveform, square waveform, sinusoidal waveform, and the like.

In operation, the wound filler 130 is inserted into the wound cavity 110 and wound cover 120 is placed so as to seal the wound cavity 110. The pump assembly 150 provides a source of a negative pressure to the wound cover 120, which is transmitted to the wound cavity 110 via the wound filler 130. Fluid (such as, wound exudate) is drawn through the conduit 140, and can be stored in a canister. In some cases, fluid is absorbed by the wound filler 130 or one or more absorbent layers (not shown).

Wound dressings that may be utilized with the pump assembly and systems of the present application include Renasys-F, Renasys-G, Renasys AB, and Pico Dressings available from Smith & Nephew. Further description of such wound dressings and other components of a negative pressure wound therapy system that may be used with the pump assembly and systems of the present application are found in U.S. Patent Publication Nos. 2012/0116334, 2011/0213287, 2011/0282309, 2012/0136325 and U.S. Pat. No. 9,084,845, each of which is incorporated by reference in its entirety. In some cases, other suitable wound dressings can be utilized.

### Pump Assembly and Canister

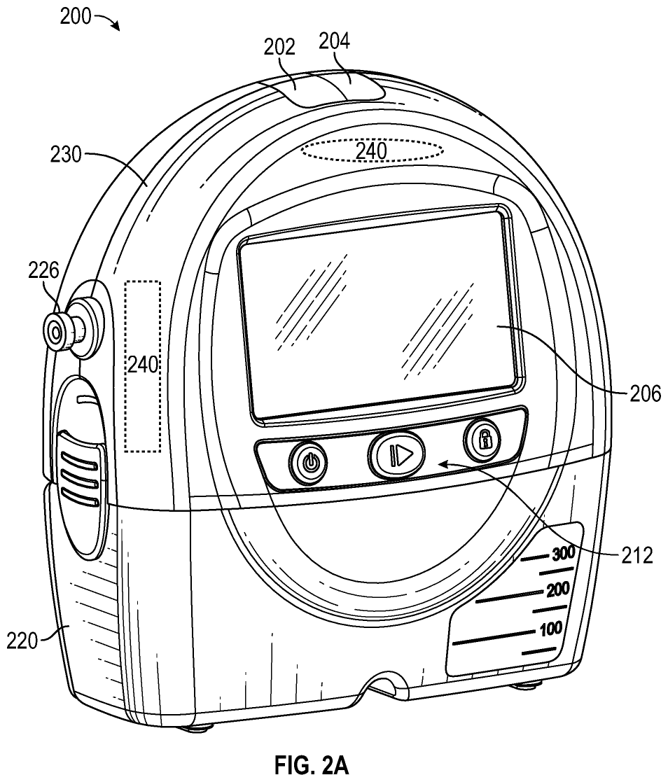

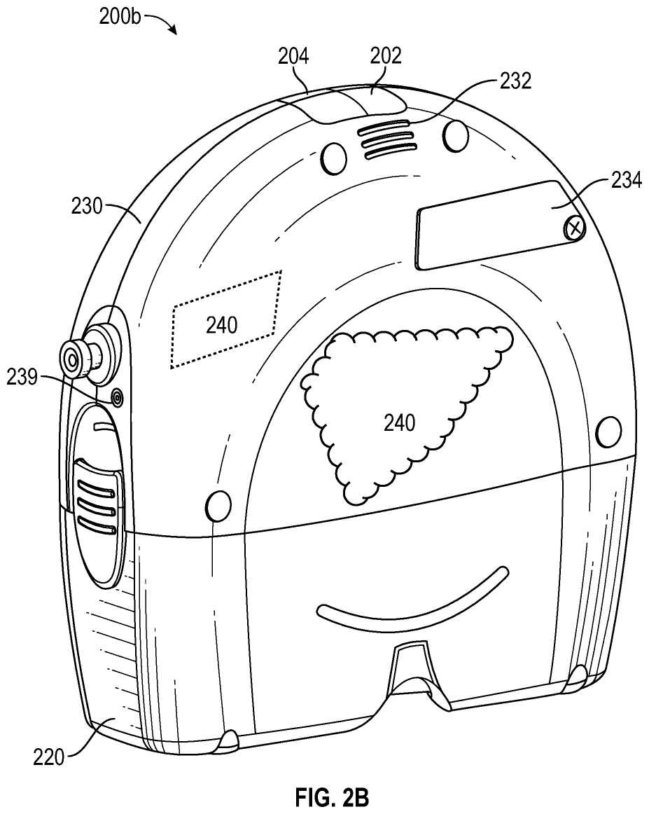

FIGS. 2A-B illustrates a negative pressure wound therapy device 200 including a pump assembly 230 and canister 220. As is illustrated, the pump assembly 230 and the canister are connected, thereby forming the device 200. The pump assembly 230 comprises one or more indicators, such as visual indicator 202 configured to indicate alarms and visual indicator 204 configured to indicate status of the TNP system. The indicators 202 and 204 can be configured to alert a user (for example, patient, health care provider, or the like) to a variety of operating and/or failure conditions of the system, including alerting the user to normal or proper operating conditions, pump failure, power supplied to the pump or power failure, detection of a leak within the wound cover or flow pathway (sometimes referred to as fluid flow path), suction blockage in the flow pathway, canister full, overpressure, or any other similar or suitable conditions or combinations thereof. In some cases, any one or more of the indicators 202 and 204 can be configured to alert a user that the current operation is compliant or non-compliant with a therapy prescription, which can be stored in a remote computing device (sometimes referred to a “remote computing system” or “remote computer”). The remote computing device can be any one or more computing devices with at least one processor and/or database, such as one or more cloud servers (sometimes referred to as “the cloud”), one or more mobile phones, one or more tablets, one or more laptops, one or more computers, one or more other negative pressure wound therapy devices, one or more therapy monitoring devices (such as, for example, described in International Patent Application No. PCT/EP2020/072663, filed on Aug. 12, 2020, which is incorporated by reference in its entirety), or the like. In some cases, the pump assembly 230 can comprise additional indicators. In some cases, a single indicator is used. In some cases, multiple indicators are used. Any one or more suitable indicators can be used such as visual, audio, tactile indicator, and so on. The indicator 202 can be configured to signal alarm conditions, such as canister full, power low, conduit 140 disconnected, seal broken in the wound seal 120, and so on. The indicator 202 can be configured to display red flashing light to draw user's attention. The indicator 204 can be configured to signal status of the TNP system, such as therapy delivery is ok, leak detected, and so on. The indicator 204 can be configured to display one or more different colors of light, such as green, yellow, etc. For example, green light can be emitted when the TNP system is operating properly and yellow light can be emitted to indicate a warning.

The pump assembly 230 comprises a display or screen 206 mounted in a recess formed in a case of the pump assembly. In some cases, the display 206 can be a touch screen display. In some cases, the display 206 can support playback of audiovisual (AV) content, such as instructional videos. As explained below, the display 206 can be configured to render a number of screens or graphical user interfaces (GUIs) for configuring, controlling, and monitoring the operation of the TNP system. The pump assembly 230 comprises a gripping portion formed in the case of the pump assembly. The gripping portion can be configured to assist the user to hold the pump assembly 230, such as during removal of the canister 220. The pump assembly 230 includes one or more strap mounts for connecting a carry strap to the pump assembly 230 or for attaching a cradle. In some cases, the canister 220 can be replaced with another canister, such as when the canister 220 has been filled with fluid.

The pump assembly 230 comprises one or more keys or buttons 212 configured to allow the user to operate and monitor the operation of the TNP system. As is illustrated, there can be a plurality of buttons. One button can be configured as a power button to turn on/off the pump assembly 230. Another button can be configured as a play/pause button for the delivery of negative pressure therapy. For example, pressing the button can cause therapy to start, and pressing the button afterward can cause therapy to pause or end. A button can be configured to lock the display 206 and/or the buttons 212. For instance, a button can be pressed so that the user does not unintentionally alter the delivery of the therapy. In some cases, multiple key presses and/or sequences of key presses can be used to operate the pump assembly 230.

The canister 220 is configured to hold fluid (such as, exudate) removed from the wound cavity 110. The canister 220 includes one or more latches for attaching the canister to the pump assembly 230. The exterior of the canister 220 can formed from frosted plastic so that the canister is substantially opaque and the contents of the canister and substantially hidden from plain view. The canister 220 includes a substantially transparent window, which can also include graduations of volume. For example, the illustrated 300 mL canister 220 includes graduations of 50 mL, 100 mL, 150 mL, 200 mL, 250 mL, and 300 mL. In some cases, the canister can hold different volume of fluid and can include different graduation scale. The canister 220 comprises a tubing channel for connecting to the conduit 140.

FIG. 2B illustrates a rear view 200B of the pump assembly 230 and canister 220. The pump assembly 230 comprises a speaker 232 for producing sound. The speaker 232 can be used to generate an acoustic alarm in response to deviations in therapy delivery, non-compliance with therapy delivery, or any other similar or suitable conditions or combinations thereof. The speaker 232 can be used to generate audio feedback to user input.

The pump assembly 230 can include a filter access door 234 for accessing and replacing one or more filters, such as antibacterial filters. The pump assembly 230 can comprise a power jack 239 for charging and recharging an internal battery of the pump assembly. In some cases, the power jack 239 is a direct current (DC) jack. In some cases, the pump assembly can comprise a disposable power source, such as batteries, so that no power jack is needed. In some cases, one of the power supplies (primary) can deliver power to operate and control the pump. In some cases, a secondary power source can deliver power to one or more of the user interface, alert system, and/or communication system for uploading usage data to the cloud.

The pump assembly 230 can include one or more areas or regions 240 as illustrated in FIGS. 2A-2B. The one or more regions 240 can correspond to locations or areas for pairing with the pump assembly 230 as described herein. The one or more regions 240 can be located on one or more of the front, side, back, top, or bottom surfaces of the pump assembly 230 and/or the canister 220. As shown, each of the one or more regions 240 can be shaped as a square, rectangle, circle, ellipsis, rhombus, parallelepiped, or have irregular shape. Each of the one or more regions can be of different size or shape than any of the other regions 240. In some cases, two or more regions 240 can be of same or similar size and shape.

### Control System

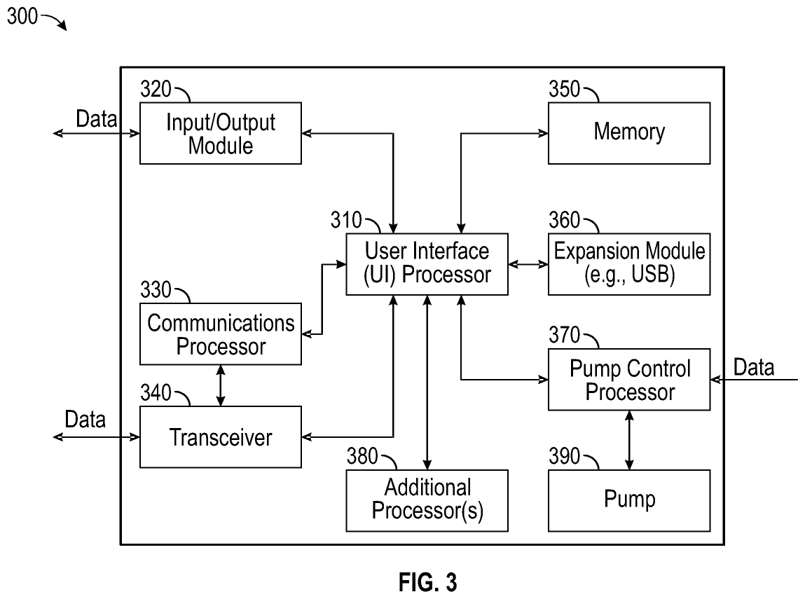

FIG. 3 illustrates a schematic of a control system 300 which can be employed in any of the embodiments of wound monitoring and/or treatment systems described herein, such as in the device 200 of FIGS. 2A-2B. Electrical components can operate to accept user input, provide output to the user, operate the negative pressure source of a TNP system, provide network connectivity, and so on. It may be advantageous to utilize multiple processors in order to allocate or assign various tasks to different processors. In some cases, a first processor can be responsible for user activity and a second processor can be responsible for controlling another device, such as a pump 390. This way, the activity of controlling the other device, such as the pump 390, which may necessitate a higher level of responsiveness (corresponding to higher risk level), can be offloaded to a dedicated processor and, thereby, will not be interrupted by user interface tasks, which may take longer to complete because of interactions with the user.

Input and output to the other device, such as a pump 390, one or more sensors (for example, one or more pressure sensors configured to monitor pressure in one or more locations of the fluid flow path), or the like, can be controlled by an input/output (I/O) module 320. For example, the I/O module can receive data from one or more sensors through one or more ports, such as serial (for example, I2C), parallel, hybrid ports, and the like.

The processor 310 can also receive data from and provide data to one or more expansion modules 360, such as one or more USB ports, SD ports, Compact Disc (CD) drives, DVD drives, FireWire ports, Thunderbolt ports, PCI Express ports, and the like. The processor 310, along with other controllers or processors, can store data in one or more memory modules 350, which can be internal and/or external to the processor 310. Any suitable type of memory can be used, including volatile and/or non-volatile memory, such as RAM, ROM, magnetic memory, solid-state memory, Magnetoresistive random-access memory (MRAM), and the like.

In some cases, the processor 310 can be a general purpose controller, such as a low-power processor. In other cases, the processor 310 can be an application specific processor. In some cases, the processor 310 can be configured as a “central” processor in the electronic architecture of the system 300, and the processor 310 can coordinate the activity of other processors, such as a pump control processor 370, communications processor 330, and one or more additional processors 380. The processor 310 can run a suitable operating system, such as a Linux, Windows CE, VxWorks, etc.

The pump control processor 370 (if present) can be configured to control the operation of a negative pressure pump 390. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. In some cases, the pump control processor 370 can measure pressure in a fluid flow path, using data received from one or more pressure sensors, calculate the rate of fluid flow, and control the pump. In some cases, the pump control processor 370 controls the pump motor so that a desired level of negative pressure in achieved in the wound cavity 110. The desired level of negative pressure can be pressure set or selected by the user. The pump control processor 370 can control the pump (for example, pump motor) using pulse-width modulation (PWM). A control signal for driving the pump can be a 0-100% duty cycle PWM signal. The pump control processor 370 can perform flow rate calculations and detect alarms. The pump control processor 370 can communicate information to the processor 310. The pump control processor 370 can include internal memory and/or can utilize memory 350. The pump control processor 370 can be a low-power processor.

A communications processor 330 can be configured to provide wired and/or wireless connectivity. The communications processor 330 can utilize one or more transceivers 340 for sending and receiving data. The one more transceivers 340 can include one or more antennas, optical sensors, optical transmitters, vibration motors or transducers, vibration sensors, acoustic sensors, ultrasound sensors, or the like. In some cases, the communications processor 330 can provide one or more of the following types of connections: Global Positioning System (GPS), cellular connectivity (for example, 2G, 3G, LTE, 4G, 5G, or the like), near field communication (NFC), Bluetooth connectivity, radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software and/or firmware, pairing, and the like.

In some cases, the communications processor 330 can provide dual GPS/cellular functionality. Cellular functionality can, for example, be 3G, 4G, or 5G functionality. In such cases, if the GPS module is not able to establish satellite connection due to various factors including atmospheric conditions, building or terrain interference, satellite geometry, and so on, the device location can be determined using the 3G network connection, such as by using cell identification, triangulation, forward link timing, and the like. In some cases, the system 300 can include a SIM card, and SIM-based positional information can be obtained. The communications processor 330 can communicate information to the processor 310. The communications processor 330 can include internal memory and/or can utilize memory 350. The communications processor 330 can be a low-power processor.

In some cases, the system 300 can store data illustrated in Table 1. This data can be stored, for example, in memory 350. This data can include patient data collected by one or more sensors. In various cases, different or additional data can be stored by system 300. In some cases, location information can be acquired by GPS or any other suitable method, such as cellular triangulation, cell identification forward link timing, and the like.

TABLE 1
Example Data Stored

| Category | Item | Type | Source |
|---|---|---|---|
| GPS | Location | Latitude, Longitude, Altitude | Acquired from GPS |
| | Timestamp Location Acquired | Timestamp | |
| Therapy | Total time therapy ON since device activation | Minutes | Calculated on device |
| | Total time therapy ON since last maintenance reset | Minutes | based on user control |
| | Device Placement; accumulated daily hours | Minutes | |
| | starting from first Therapy ON after last | | |
| | maintenance reset, stopping at last Therapy OFF | | |
| | before returning for Maintenance and maintenance | | |
| | reset. (Includes both THERAPY ON and THERAPY | | |
| | OFF hours) | | |
| Device | Serial Number | Alphanumeric | Set by Pump Utility |
| | Controller Firmware Version | Alphanumeric | Unique version |
| | | | identifier, hard coded in |
| | | | firmware |
| Events | Device Event Log (See Table 3 for example) | List of Events (See Table 2) | Generated in response to |
| | | | various user actions and |
| | | | detected events |

The system 300 can track and log therapy and other operational data. Such data can be stored, for example, in the memory 350. In some cases, the system 300 can store log data illustrated in Table 2. Table 3 illustrates an example event log. One or more such event logs can be stored by the system 300. As is illustrated, the event log can include time stamps indicating the time of occurrence of a particular event. For example, Table 3 illustrates that the negative pressure source was activated at 1:31:02 UTC on Apr. 22, 2012 to provide therapy at a set point of −120 mmHg (with medium ramp-up intensity) in a continuous mode of operation and that at 1:44:20 UTC on the same day a high flow leak was detected and therapy was stopped at 1:44:24 UTC on the same day. In some cases, additional and/or alternative data can be logged.

TABLE 2
Example Data Tracked

| Category | ID | Type | Data Content | Notes |
|---|---|---|---|---|
| Device | 0 | Startup (Created DB) | | First time, out-of-the-box. |
| | 1 | Startup (Resumed DB) | | Subsequent power-ups. |
| | 2 | Startup (Corrupt DB, Recreated) | | Corrupt configuration was detected. The |
| | | | | database was deleted and recreated, and |
| | | | | next run was in out-of-the-box mode. |
| | 3 | Shutdown (Signaled) | | Normal shutdown, handled/registered by |
| | | | | software. |
| | 4 | Shutdown (Inferred) | | Unexpected shutdown; on next power-up, |
| | | | | last active time registered as shutdown |
| | | | | event. |
| Therapy | 5 | Start Delivery (Continuous) | modes, setpoints | Modes are Y-connect status, and intensity. |
| | 6 | Start Delivery (Intermittent) | modes, setpoints | Modes are Y-connect status, and intensity. |
| | 7 | Stop Delivery | | |
| | 8 | Set Therapy Pressure Setpoint | mmHg | This and other therapy adjustment events |
| | | | | are only recorded while therapy is being |
| | | | | delivered. |
| | 9 | Set Standby Pressure Setpoint | mmHg | |
| | 10 | Set Intermittent Therapy Duration | setting (30 s, 60 s, etc) | |
| | 11 | Set Intermittent Standby Duration | setting (30 s, 60 s, etc) | |
| | 12 | SetMode | cont/intermittent | |
| | 13 | Set Intensity | low/med/high | |
| | 14 | Set Y Connect | yes/no | |
| Alarm | 15 | Over Vacuum | high mmHg | |
| | 16 | High Vacuum | high deviation mmHg | |
| | 17 | Blocked Full Canister | low airflow lpm | |
| | 18 | High Flow Leak | high airflow lpm | |
| | 19 | Low Vacuum | low mmHg | |
| | 20 | Battery Failure | | |
| | 21 | Critical Battery | | |
| | 22 | Low Battery | | |
| | 23 | Inactivity | | |
| Maintenance | 24 | Maintenance Reset | | |
| | 25 | Reset to Defaults | | |
| | 26 | Software/Device Warning | Warning code | Any detected, minor unexpected software |
| | | | | behavior will be logged as an event |
| | 27 | Software/Device Fault | Fault code | Any detected, severe unexpected software |
| | | | | behavior will be logged as an event |

TABLE 3
Example Event Log

| | Type | | | |
|---|---|---|---|---|
| Timestamp | ID | Type Description | Data | |
| 1:23:45 4/2/2012 (UTC-12) | 0 | Startup (Created DB) | | |
| 1:29:23 4/2/2012 (UTC-12) | 15 | Set Intensity | medium | |
| 1:29:43 4/2/2012 (UTC-12) | 10 | Set Therapy Pressure Setpoint | 120 | mmHg |
| 1:31:02 4/2/2012 (UTC-12) | 7 | Start Delivery (Continuous) | 120 mmHg continuous, | |
| | | | medium intensity, no Y | |
| | | | connect | |
| 1:44:20 4/2/2012 (UTC-12) | 20 | High Flow Leak | 4 | lpm |
| 1:44:24 4/2/2012 (UTC-12) | 9 | Stop Delivery | | |

In some cases, using the connectivity provided by the communications processor 330, the system 300 can upload any of the data stored, maintained, and/or tracked by the system 300 to a remote computing device. In some cases, the following information can be uploaded to the remote computing device: activity log(s), which includes therapy delivery information, such as therapy duration, alarm log(s), which includes alarm type and time of occurrence; error log, which includes internal error information, transmission errors, and the like; therapy duration information, which can be computed hourly, daily, and the like; total therapy time, which includes therapy duration from first applying a particular therapy program or programs; lifetime therapy information; device information, such as the serial number, software version, battery level, etc.; device location information; patient information; and so on. The system 300 can also download various operational data, such as therapy selection and parameters, firmware and software patches and upgrades, and the like. The system 300 can provide Internet browsing functionality using one or more browser programs, mail programs, application software (for example, apps), etc. Additional processors 380, such as processor for controlling one or more user interfaces (such as, one or more displays), can be utilized. In some cases, any of the illustrated and/or described components of the system 300 can be omitted depending on an embodiment of a wound monitoring and/or treatment system in which the system 300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Canisterless Pump Assembly

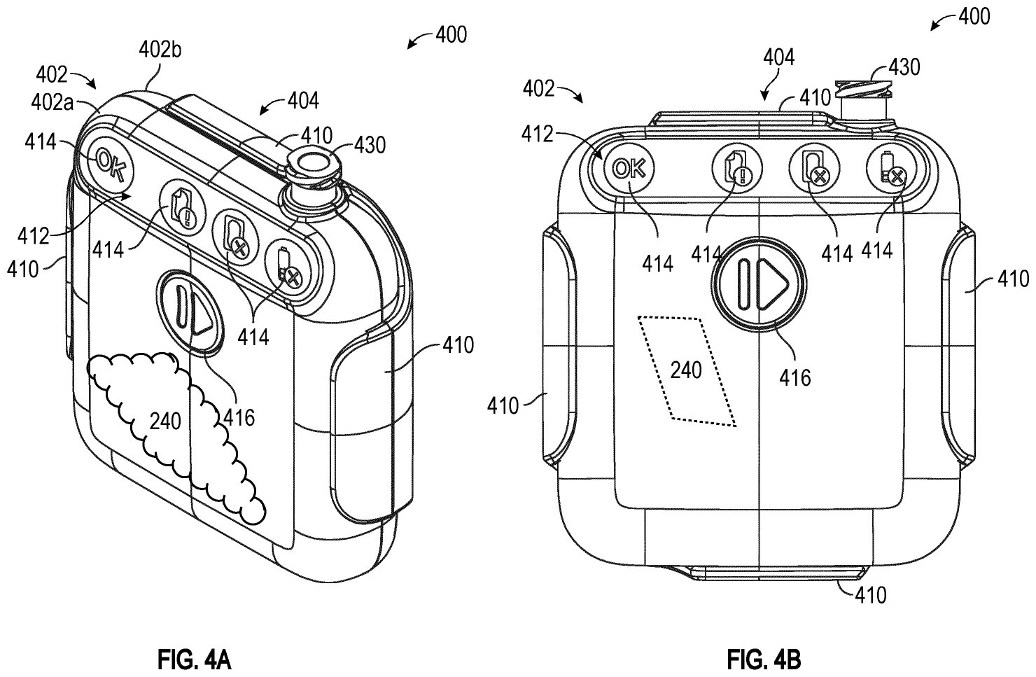

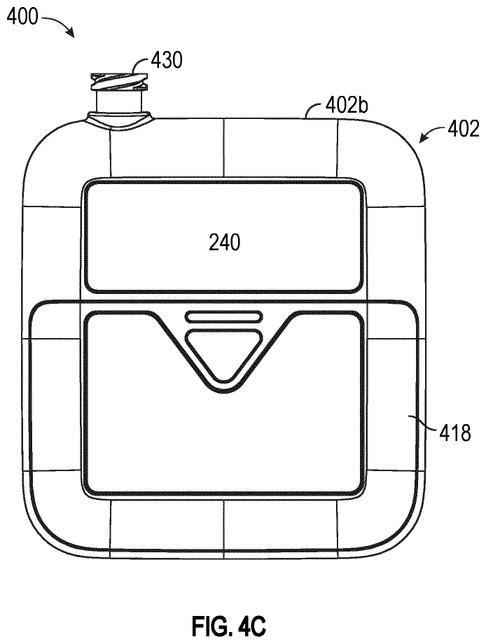

FIGS. 4A-4C illustrate perspective, front, and rear views of a reduced pressure wound therapy device 400. The reduced pressure wound therapy device 400 can include a housing 402 and an optional mounting component 410. The mounting component (or attachment) 410 can be removably attached to the housing 402, such that the reduced pressure wound therapy device 400 can be used with or without the mounting component 410. For example, FIG. 4C illustrates the reduced pressure wound therapy device 400 without the mounting component 410. The mounting component 410 can be designed to allow the reduced pressure wound therapy device 400 to be mounted on another object such as, but not limited to, a user's person. The mounting component 410 can include a clip designed to retain the mounting component 410 on a user's outerwear, such as on a user's pocket, a pouch, a belt, a flap, or otherwise.

The housing 402 (sometimes referred to as “outer housing”) can contain or support components of device reduced pressure wound therapy device 400. The housing 402 can be formed from one or more portions, such as a front portion 402a and a rear portion 402b, which can be removably attached to form the housing 402.

The housing 402 can include a user interface 412 which can be designed to provide a user with information (for example, information regarding an operational status of the reduced pressure wound therapy device 400). The user interface 412 can include one or more indicators, such as icons 414, which can alert the user to one or more operating or failure conditions of the reduced pressure wound therapy system. For example, the indicators can include icons for alerting the user to normal or proper operating conditions, pump failure, power failure, the condition or voltage level of the batteries, the condition or capacity of a wound dressing, detection of a leak within the wound dressing or fluid flow pathway between the wound dressing and the pump assembly, suction blockage, or any other similar or suitable conditions or combinations thereof. An example set of icons 414 is illustrated in FIGS. 4A-4B which, from left to right, can include an “OK” indicator which can indicate normal operation of the system, a “leak” indicator which can indicate the existence of a leak in the system, a “dressing full” indicator which can indicate that a wound dressing is at or near capacity, and a “battery critical” indicator which can indicate that the power source (such as, one or more batteries) is at or near a critical level. The icons 414 can have a green or orange color, or can be illuminated with a green or orange light (for example, colored LEDs).

The reduced pressure wound therapy device 400 can include one or more user input features, such as button 416, designed to receive an input from the user for controlling the operation of the device 400. A single button can be present which can be used to activate and deactivate the reduced pressure wound therapy device or control other operating parameters of the device 400. For example, the button 416 can be used to activate the reduced pressure wound therapy device 400, pause the device 400, clear indicators (such as, one or more icons 414, or be used for any other suitable purpose for controlling an operation of the device 400 (for example, by sequentially pushing on the button 416). The button 416 can be a push style button that can be positioned on an outside, front surface of the housing 402. In some cases, multiple input features (for example, multiple buttons) can be provided.

The reduced pressure wound therapy device 400 can include a connector 430 for connecting a tube or conduit to the device 400. The connector 430 can be used to connect reduced pressure wound therapy device to a wound dressing.

The reduced pressure wound therapy device 400 can be a canisterless device. The wound dressing can retain fluid (such as, exudate) aspirated from the wound. Such a dressing can include a filter, such as a hydrophobic filter, that prevents passage of liquids downstream of the wound dressing (toward the reduced pressure wound therapy device 400).

The reduced pressure wound therapy device 400 can include a removable cover 418, as illustrated in FIG. 4C. The cover 418 can cover a cavity (not shown) in which one or more power sources, such as batteries, for powering the device 400 are positioned.

As illustrated in FIGS. 4A-4C, the reduced pressure wound therapy device can include one or more regions 240, which can function as described herein.

Any of the negative pressure wound therapy devices described herein can include one or more features disclosed in U.S. Patent Publication No. 2019/0231939, which is incorporated by reference in its entirety.

Pairing with Therapy Device

It may be advantageous to be able to remotely communicate with any of the reduced pressure wound therapy devices described herein, such as the devices 200 or 400. As described herein, such remote communication can be used for one or more of retrieving therapy data, operational data, location data, or the like from a reduced pressure wound therapy device, controlling operation of the reduce pressure wound therapy device (for example, programing the device), or the like. Remote communication with the reduced pressure wound therapy device can be performed over a wireless connection. In case of a reduced pressure wound therapy device having a limited user interface (such as, the user interface 412 of the device 400 that includes one or more icons), remote communication with the reduced pressure wound therapy device can advantageously allow the user to access additional data relating to the operation of the reduced pressure wound therapy device in addition to the data accessible through the user interface.

Remote communication can be established between a remote computing device and the reduced pressure wound therapy device. As described herein, the remote computing device can be a mobile phone, tablet, laptop, computer, another reduced pressure wound therapy device, or the like. In some cases, remote communication can be accomplished using a Bluetooth communication protocol, such as Bluetooth (for instance, Bluetooth 4.0, 4.1, 4.2, 5, 5.1, BLE (Bluetooth low energy), etc.), Wi-Fi communication protocol, Zigbee communication protocol, Z-Wave communication protocol, WLAN communication protocol, WPAN communication protocol, or the like. For security and integrity, including protection of patient medical data, ensuring that correct remote computing device is connected to correct reduced pressure wound therapy device (as there may be many remote computing devices in a vicinity of a reduce pressure wound therapy device and vice versa), or the like, it can be advantageous to pair or bond a remote computing device with the reduced pressure wound therapy device. Pairing can be performed before or as part of establishing the remote communication.

In some cases, pairing can be performed using “just works” approach by which a known key (for example, 0 or another suitable value) is exchanged between the reduced pressure wound therapy device and the remote computing device. The temporary key can then be used to establish the remote communication, encrypt the remote communication, or the like. While this approach may not require user interaction, it may not be secure and/or facilitate connection between the correct devices, or the like. These shortcoming may be due to the key being known.

In some cases, pairing can be performed using “passkey” approach by which a key is passed between the remote computing device and the reduced pressure wound therapy device by the user. For example, a random key can be generated and displayed on the reduced pressure wound therapy device (or on the remote computing device). The user can enter the key into the remote computing device (or into the reduce pressure wound therapy device). The key can then be used to establish the remote communication, encrypt the remote communication, or the like. While this approach may facilitate security, connection between the correct devices, or the like, it may undesirably require user interaction.

In some cases, pairing can be performed by exchanging pairing data between the remote computing device and the reduced pressure wound therapy device using a first communication protocol different from a second communication protocol that is used for one or more of retrieving therapy data, operational data, location data, or the like data from a reduced pressure wound therapy device, controlling operation of the reduce pressure wound therapy device (for example, programming the device), or the like. For example, a key can be generated and exchanged between the remote computing device and the reduced pressure wound therapy device using inductive coupling, NFC, RFID (for example, passive RFID), optical communication, or the like. As another example, an IP address (and/or credentials) can be exchanged to establish a Wi-Fi connection, which may be used to then establish a Bluetooth connection. This can be performed when the remote computing device is positioned in physical contact with or within a threshold distance of one or more regions of the reduced pressure wound therapy device (such as, the one or more regions 240). The key (or IP address) can be randomly generated. The key (or IP address) can be used to establish the remote communication, encrypt the remote communication, or the like. The remote communication can utilize Bluetooth, Wi-Fi, or the like.

Advantageously, this approach can facilitate security, connection between the correct devices, etc. while not requiring any user interaction or requiring minimal user interaction. In some cases, first and second communication protocols can be the same.

In some cases, the user may be requested to authorize or confirm the pairing. This can be done by pressing a button of the reduced pressure therapy device, such as any of buttons 212, touching the display 206, or the like. Alternatively or additionally, the user can authorize the pairing on the remote computing device.

With reference to FIGS. 2A-2B and 4A-4C, any of the one or more regions, such as regions 240, on the surface of the housing of the reduced pressure wound therapy device can correspond to coverage area(s) of one or more antennas configured to facilitate the pairing using the first communication protocol. Coverage area of an antenna can be associated with space in which the antenna is configured to receive and/or transmit radio frequency (RF) waves or signals. For example, one or more of an antenna for inductive coupling, NFC antenna, RFID antenna (such as, passive RFID antenna), or the like can be positioned within the housing adjacent to or proximal to any of the regions 240. For example, an antenna can be positioned within the housing below the surface of a region 240. Coverage area of the antenna used for pairing can be limited to include the area enclosed by the region 240 and/or volume extending a threshold distance away from the area enclosed by the region 240. The threshold distance can a small distance away from the surface of the housing, such as 0.5 cm or less, 1 cm or less, 2 cm or less or more, 3 cm or less or more, 4 cm or less or more, 5 cm or less or more, 10 cm or less or more, 15 cm or less or more. Unless the remote computing device is positioned within the coverage area of the antenna (such as, in contact with the area enclosed by the region or within the volume defined by the region 240 and the threshold distance), pairing will not be initiated. Limiting the coverage areas of the one or more antennas can improve security (for example, by preventing hacking) and promote connection between the correct devices. In some cases, to further promote security the one or more regions may not be marked by any indicia (such that, the user may need to a priori know the location(s) of the one or more regions to initiate the pairing).

Coverage area of the antenna can be limited by using a directional antenna. For example, a directional inductive coupling, NFC, RFID, or the like antenna can be used. The directional antenna can be a short range antenna. As another example, coverage area of the antenna can be limited by reducing the dimensions of the antenna (such as, removing one or more coils from a loop antenna, decreasing antenna size, or the like). As yet another example, the housing can be thinner in a region 240 as compared to portions of the housing adjacent to the region 240 (for example, surrounding the region 240). The antenna may be able to transmit and receive RF waves due in the region where the housing is thinner, but not in the other regions because thicker housing may block the RF waves. As yet another example, an antenna can be at least partially shielded by an electromagnetic shield. For instance, the antenna can be at least partially enclosed in a Faraday cage or shield. This can limit the coverage area of the antenna to a near field region, which can span about a wavelength of the RF wave or a fraction of the wavelength. Limiting the coverage area to the near field region can be suitable for inductive coupling communication, NFC communication, RFID communication, or the like. In some cases, any one or more of the approaches for limiting the coverage area of an antenna can be combined.

In some cases, pairing can include positioning the remote computing device in the coverage areas of multiple antennas configured for pairing (or positioning the remote computing device in the coverage area of the same antenna multiple times). For example, as shown in FIGS. 2A-2B and 4A-4C, there can be a plurality of regions 240 associated with a plurality of antennas configured for pairing. Pairing may be accomplished by positioning the remote computing device within a coverage area of a first antenna associated with a first region 240 and subsequently positioning the remote computing device within a coverage area of a second antenna associated with a second region 240. Portions of pairing data can be exchanged between the devices in response to the remote computing device being positioned within the coverage area of the first and second antenna. Alternatively or additionally, in response to the remote computing device being positioned within expected sequence of coverage areas of the plurality of antennas, entire pairing data can be exchanged between the devices when the remote computing device is positioned in the coverage area of the last antenna in the sequence. In some cases, the duration of time the remote computing device is positioned within the coverage area of an antenna can be used to facilitate the pairing. For example, the remote computing device may be expected to be positioned within the coverage area for a threshold time duration, such as 5 seconds or less or more, 6 seconds or less or more, 10 seconds or less or more, 15 seconds or less or more, 20 seconds or less or more, 30 seconds or less or more. In some cases, additional regions 240 (such as, third, fourth, fifth, etc.) can be used. In some cases, the same region 240 (or same set of regions 240) can be utilized, and the remote computing device can be positioned in the region 240 (or set of regions 240) for one or more durations of time. These approaches can further improve security and promote connection between the correct devices.

Pairing can involve tapping or touching a reduced pressure wound therapy device with a remote computing device (or vice versa). Tapping or touching can be detected by one or more sensors positioned, for example, in the one or more regions 240, such as, a force sensor, motion sensor (such as, accelerometer), etc. Tapping or touching to pair can be performed in sequence as described above with respect to positioning the remote computing device in the coverage areas of multiple antennas (or in the coverage area of a single antenna). In some cases, user interface of the remote computing device (and/or the reduced pressure wound therapy device) can guide the user through the sequence of tapping or touching steps.

The remote computing device can be placed in contact with the reduced pressure wound therapy device, and vibration can be used to facilitate the pairing. For example, one or more of the remote computing device or the reduced pressure wound therapy device can include one or more of a vibration motor (or vibration transducer) and/or vibration sensor (such as, accelerometer, force sensor, vibration meter, or the like). One or more vibration sensors can be positioned in the one or more regions 240. Pairing data can be exchanged by modulating the vibration signal. Pairing by using vibration can be performed in a sequence as described above with respect to positioning the remote computing device in the coverage areas of multiple antennas (or in the coverage area of a single antenna).

Optical communication can be used for pairing. One or more of the regions 240 of the reduced pressure wound therapy device can include optical (or light) sensor(s) configured to receive light emitted by optical (or light) transmitter(s) of the remote computing device. For example, one or more optical sensors can be positioned adjacent to the surface of a region 240, such as, below the exterior surface of the housing. As another example, one or more optical transmitters can be positioned adjacent to the surface of the region 240, such as, below the exterior surface of the housing. Surface of the housing within the region 240 can be at least partially optically transparent or at least partially optically translucent to permit light emitted by one or more optical transmitters of the remote computing device to be received by the one or more optical sensors. Portions of the housing adjacent to the regions 240 (for example, surrounding the region 240) can be optically opaque, thus blocking light transmission. For instance, the region 240 can include an optically transparent or translucent window. As another example, the region 240 can include a diffuser for scattering light transmitted by one or more optical transmitters.

Pairing data can be communicated using an optical communication protocol. The optical communication protocol can utilize infrared light. For instance, infrared light (such as, as specified in infrared data association (IrDA) protocol, infrared simple (IrSimple) protocol, light fidelity (Li-Fi) protocol, etc.), visible light (such as, specified in the Li-Fi protocol), ultraviolet light (such as, specified in the Li-Fi protocol), lasers, or the like can be utilized. Optical sensors can include one or more photodetectors, photodiodes, phototransistors, photoconductive sensors, photovoltaic sensors, etc. Optical transmitters can include one or more of light emitting diodes (LEDs), lamps, etc.

A latching circuit (or a latch) can be used for pairing. For example, the latching circuit can include one or more optical sensors configured to activate (or set) and/or deactivate (or reset) the latching circuit in response to receiving light. To set (and/or reset) the latching circuit, received light may need to satisfy of one or more of threshold intensity, threshold wavelength, particular sequence of wavelengths and/or intensities, or the like. Activation of the latching circuit can trigger the pairing (and deactivation of the latching circuit can trigger unpairing). This approach can utilize one or more optically transparent, translucent, or opaque regions are described herein.

One or more barcodes (such as, QR codes, text, or images) (sometimes collectively referred to as “indicia”) can be positioned or displayed, for example, in one or more regions 240 (such as, in the region 240 illustrated in FIG. 4C). Indicia can be drawn, printed, or affixed to the exterior surface of the housing (for example, within one or more regions 240), displayed electronically (such as, on a display), or the like. Indicia can include pairing data (for example, encoded pairing data). In some cases, the remote computing device can take a picture of the indicia, process the image, and retrieve the pairing data.

Internal clocks of the remote computing device and the reduced pressure wound therapy device can be synchronized during the pairing. This can be advantageous for interpreting data that is associated with particular point in time and/or is in chronological order. For example, as described herein in connection with Table 3, the reduced pressure wound therapy device can maintain an event log that includes time stamps indicating the time of occurrence of a particular event. Synchronizing with the clock of the reduced pressure wound therapy device during pairing can facilitate associating the time stamps in the event log data with the clock of the remote computing device. Synchronization can facilitate presenting to the user the correct chronological sequence of the event log data.

A reduced pressure wound therapy device can include a plurality of antennas. For example, a first antenna can be used for pairing via the first protocol. The first antenna can have a limited coverage area as described herein. A second antenna can be used for remote communication via the second protocol (such as, via Bluetooth, Wi-Fi, etc.). To facilitate remote communication over larger distances, the second antenna can have a greater coverage area than the first antenna.

A remote computing device can be another reduced pressure wound therapy device. For example, the device 200 can be used to pair with the device 400 and to remotely communicate with the device 400. Because the device 200 can include a more enhanced user interface than that of the device 400 (for example, the device 200 can include the display 206), can advantageously allow the user to access additional data relating to the operation of the device 400 in addition to the data accessible through the user interface 412.

Reduced pressure wound therapy devices can broadcast their availability of pairing. For example, Bluetooth protocol can be used for broadcasting. The user of the remote computing device can discover reduced pressure wound therapy devices located in proximity and pair with one or more of the reduced pressure wound therapy devices.

In some cases, a reduced pressure wound therapy device can be paired with multiple remote computing devices using any one or more approaches described herein.

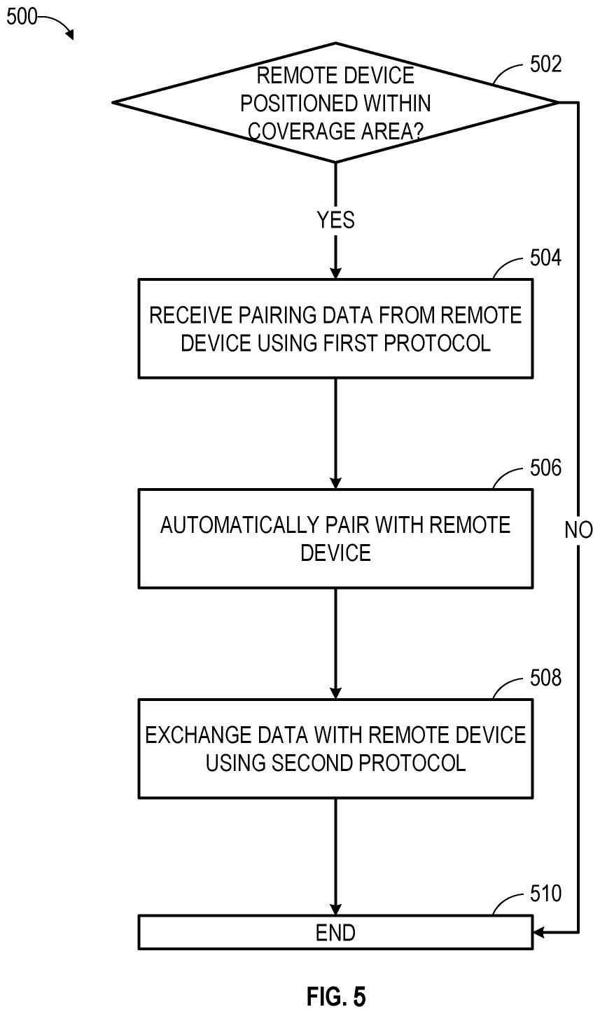

FIG. 5 illustrates a process 500 of pairing with a reduced pressure wound therapy device. The process 500 can be implemented by one or more processors of the reduced pressure wound therapy device, such as by the communications processor 330 alone or in combination with the processor 310. The process 500 can start in block 502 in which pairing data is received by the process 500. As descried herein, pairing data may be received by the process 500 only when a remote computing device is positioned within the coverage area of a reduced pressure wound therapy device antenna configured to facilitate the pairing. In addition, as described herein, coverage area can more broadly refer to area or space in which pairing data can be exchanged. Such area or space can include one or more regions facilitating optical transmission of data, transmission of data by tapping or touching, transmission of data via vibration, or the like. If the remote computing device is positioned within the coverage area, the process 500 can transition to block 504. Otherwise, the process can terminate in block 510.

In block 504, the process 500 can receive pairing data from the remote computing device using the first communication protocol. As described herein, pairing data can include one or more of a key or IP address for pairing over Bluetooth, Wi-Fi or the like, credentials (such as, login and password), etc. The process can transition to block 506 in which the pairing data can be used to pair the remote computing device with the reduced pressure wound therapy device. As described herein, pairing can be performed automatically (or nearly automatically) by requiring no user interaction (or with minimal user interaction). Once the devices have been paired, the process 500 can transition to block 508. In block 508, the process 500 can transmit and/or receive data using the second communication protocol.

In some cases, the remote computing device can be unpaired from the reduced pressure wound therapy device (or vice versa). This can be performed automatically, such as at expiration of a duration of time (such as 10 minutes or less or more, 30 minutes or less or more, 1 hour or less or more, 3 hours or less or more, 1 day or less or more), or in response to a user request. When the devices have been unpaired, the process 500 can terminate in block 510.

Any of the pairing approaches described herein can be combined. While described are some examples in which pairing is based on positioning a remote computing device within coverage area(s) of one or more antenna(s) of a reduced pressure wound therapy device, pairing can be facilitated by positioning the reduced pressure wound therapy device in coverage area(s) of one or more antenna(s) of the remote computing device.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures may be implemented as software and/or firmware on a processor, controller, ASIC, FPGA, and/or dedicated hardware. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A negative pressure wound therapy device comprising:
a housing with an exterior surface, the exterior surface of the housing comprising a region configured to facilitate pairing with a computing device, the housing enclosing a source of negative pressure and an electronic circuitry;
the source of negative pressure configured to aspirate fluid from a wound covered by a wound dressing; and
the electronic circuitry configured to control the source of negative pressure and to wirelessly transmit and receive data, the electronic circuitry further configured to:
receive pairing data from the computing device in response to the computing device physically contacting a portion of the exterior surface of the housing within the region or the computing device being positioned at a distance satisfying a threshold distance from the exterior surface of the housing within the region, the pairing data being received using a first communication protocol;
pair with the computing device using the pairing data; and
subsequent to the pairing, transmit data to and receive data from the computing device using a second communication protocol different from the first communication protocol.

2. The device of claim 1, wherein the electronic circuitry is configured to receive the pairing data in response to the computing device physically contacting the portion of the exterior surface of the housing within the region or the computing device being positioned at the distance from the exterior surface of the housing within the region satisfying the threshold distance and not in response to the computing device physically contacting any other portion of the exterior surface of the housing or the computing device being positioned at another distance from the exterior surface of the housing within the region not satisfying the threshold distance.

3. The device of claim 1, further comprising an antenna coupled to the electronic circuitry and configured to wirelessly transmit and receive data, a coverage area of the antenna comprising one or more of the region of the exterior surface of the housing or a volume extending no farther than the threshold distance away from the exterior surface of the housing within the region.

4. The device of claim 3, wherein the coverage area of the antenna does not extend to any other portion of the exterior surface of the housing outside of the region or to any other location outside the volume.

5. The device of claim 3, wherein the antenna is positioned within the housing adjacent to the exterior surface of the housing within the region, and wherein a thickness of the housing within the region is thinner than a thickness of the housing adjacent to the region.

6. The device of claim 3, further comprising an electromagnetic shield that limits the coverage area of the antenna to one or more of the region of the exterior surface of the housing or the volume extending no farther than the threshold distance away from the exterior surface of the housing within the region.

7. The device of claim 1, wherein the electronic circuitry is configured to transmit data to and receive data from the computing device only subsequent to the pairing with the computing device.

8. The device of claim 1, wherein the pairing data comprises a passcode or an IP address.

9. The device of claim 1, wherein the electronic circuitry is further configured to encrypt data transmitted over the second communication protocol using the pairing data.

10. The device of claim 1, wherein the first communication protocol comprises near-field communication (NFC) or radio frequency identification (RFID) and the second communication protocol comprises Bluetooth or Wi-Fi.

11. The device of claim 1, wherein the data transmitted to the computing device comprises one or more of operational data associated with provision of negative pressure wound therapy or location data associated with location of the device.

12. (canceled)

13. The device of claim 1, further comprising an optical sensor positioned within the housing adjacent to the exterior surface of the housing within the region, wherein:
the exterior surface of the housing within the region is at least one of optically transparent or optically translucent;
the exterior surface of the housing adjacent to the region is optically opaque; and
the first communication protocol comprises an optical communication protocol.

14. The device of claim 13, wherein the exterior surface of the housing within the region comprises a window made of optically transparent material.

15. The device of claim 1, wherein:
the exterior surface of the housing comprises a light transmitter and the first communication protocol comprises an optical communication protocol, and/or
the exterior surface of the housing comprises a barcode that includes the pairing data.

16. (canceled)

17. A method of operating a negative pressure wound therapy device, the method comprising, by an electronic circuitry of the negative pressure wound therapy device:
receiving pairing data from a remote computing device in response to the remote computing device physically contacting a portion of an exterior surface of a housing of the negative pressure wound therapy device within a region configured to facilitate pairing with the remote computing device or the remote computing device being positioned at a distance satisfying a threshold distance from the exterior surface of the housing within the region, the pairing data being received using a first communication protocol;
pairing with the remote computing device using the pairing data; and
subsequent to the pairing, transmitting data to and receiving data from the remote computing device using a second communication protocol different from the first communication protocol.

18. The method of claim 17, further comprising, by the electronic circuitry, receiving the pairing data in response to the remote computing device physically contacting the portion of the exterior surface of the housing within the region or the remote computing device being positioned at the distance from the exterior surface of the housing within the region satisfying the threshold distance and not in response to the remote computing device physically contacting any other portion of the exterior surface of the housing or the remote computing device being positioned at another distance from the exterior surface of the housing within the region not satisfying the threshold distance.

19. The method of claim 17, wherein the negative pressure wound therapy device comprises an antenna with a coverage area including one or more of the region of the exterior surface of the housing or a volume extending no farther than the threshold distance away from the exterior surface of the housing within the region, and wherein the coverage area of the antenna does not extend to any other portion of the exterior surface of the housing outside of the region or to any other location outside the volume.

20. (canceled)

21. The method of claim 19, wherein the antenna is positioned within the housing adjacent to the exterior surface of the housing within the region, and wherein a thickness of the housing within the region is thinner than a thickness of the housing adjacent to the region.

22. The method of claim 19, wherein the antenna is at least partially shielded by an electromagnetic shield that limits the coverage area of the antenna to one or more of the region of the exterior surface of the housing or the volume extending no farther than the threshold distance away from the exterior surface of the housing within the region.

23. The method of claim 19, wherein the negative pressure wound therapy device comprises at least one of:
an optical sensor positioned within the housing adjacent to the exterior surface of the housing within the region, wherein:
the exterior surface of the housing within the region is at least one of optically transparent or optically translucent;
the exterior surface of the housing adjacent to the region is optically opaque; and
the first communication protocol comprises an optical communication protocol, or
a barcode positioned on the exterior surface of the housing, the barcode including the pairing data.
